(19) United States
(12) Patent Application Publication
(10) Pub. No.: US 2005/0181071 A1
(43) Pub. Date: Aug. 18, 2005

(54) Method for the treatment of clinical depression

(76) Inventor: Michael Raymond Binder, Highland Park, IL (US)

Correspondence Address: MICHAEL R. BINDER, MD.;SUITE 210, 767 PARK AVENUE WEST, HIGHLAND PARK, IL, 60035, US

(21) Appl. No.: 11/058,661

(22) Filed: Feb. 15, 2005

Related U.S. Application Data
(60) Provisional application No. 60/545,223, filed on Feb. 18, 2004.
(60) Provisional application No. 60/581,627, filed on Jun. 22, 2004.

(51) Int. Cl. A61K 31/5513; A61K 31/55; A61K 31/195; A61K 31/19

(52) U.S. Cl. 424/722; 514/217; 514/221; 514/561; 514/557; 514/389

(57) ABSTRACT

This invention is a new method for the treatment of clinical depression. Specifically, the invention involves reducing neurotransmission in the limbic system of the human brain as a means of treating depressive symptoms.

### BACKGROUND OF THE INVENTION

Clinical depression is among the most common illnesses of our time, yet remains one of the most challenging to treat. Many patients are unresponsive to antidepressants, and others require repeated dosage adjustments in order to sustain improvement. The currently recognized forms of clinical depression include major depressive disorder, dysthymia, seasonal affective disorder, and postpartum depression. All of these disorders are characterized by signs and symptoms such as depressed mood, low energy, poor concentration, loss of interest in pleasurable activities, alterations in sleep, changes in weight, psychomotor agitation or retardation, feelings of worthlessness, and morbid thinking. Diagnostic and Statistical Manual of Mental Disorders, Fourth Edition (DSM-IV) criteria allow for a fluctuation in some of these symptoms, but not into the range of mania or hypomania. If there is a history of mania or hypomania, the syndrome falls into a different diagnostic category, known as bipolar disorder. The pathophysiology and treatment of bipolar disorder are different than for unipolar depression, and lie outside the scope of this invention.

The currently held belief is that unipolar depression is the result of an abnormal reduction of monoamine transmission in the limbic system of the brain [1]. Hence, unipolar depression is customarily treated with medications that increase monoamine transmission, such as serotonin reuptake inhibitors (SSRIs), tricyclic antidepressants (TCAs), and monoamine oxidase inhibitors (MAOIs).

The present invention is based upon the novel idea that depressive symptoms in unipolar depression are the result of increased, rather than decreased monoamine transmission in the limbic system. Functional imaging studies suggest that human emotions are linked to specific neurochemical pathways in the brain [2-10]. This leads to the hypothesis that depressive symptoms are caused by hyperexcitability in the neuronal pathways that correspond to them. According to the multipathway neuronal hyperexcitability (MPNH) hypothesis, neuronal hyperexcitability in dysphoria-producing pathways leads to a relative rise in neurotransmission in those pathways, which, in turn, creates feelings of depression. This is the converse of the currently held hypothesis, which contends that depressive symptoms are the result of an abnormal reduction in monoamine transmission.

If the MPNH hypothesis is correct, and preliminary clinical evidence suggests that it is, depressive symptoms could be treated by reducing neurotransmission in dysphoria-producing pathways. This would be an entirely new strategy in the treatment of clinical depression.

In conjunction with the MPNH hypothesis, the invention employs various mechanisms by which to reduce neurotransmission in dysphoria-producing pathways and, thus, to reduce depressive symptoms.

1. A novel approach to the treatment of clinical depression in which the neuropathology is recognized as a relative excess of monoamine transmission in dysphoria-producing pathways in the brain, thus pointing to the need to achieve a relative reduction of neurotransmission in these pathways. This can be accomplished by one or a combination of methods.

2. A method according to claim 1 wherein symptoms of depression are treated with medications that reduce neurotransmission by reducing neuronal excitability or otherwise “stabilizing” neuronal membranes. Such medications include, but are not limited to pregabalin and its derivatives, valproic acid (Depakene, Depakote, Depakote ER), carbamazepine (Tegretol, Carbatrol), oxcarbamazepine (Trileptal), gabapentin (Neurontin), topamax (Topiramate), lamotrigine (Lamictal), levetiracetam (Keppra), tiagabine (Gabitril), zonesamide (Zonagran), ethusoximide (Zarontin), primidone (Mysolin), phenytoin (Dilantin), clonazepam (Klonopin), diazepam (Valium), chlordiazepoxide HCL (Librium), and lithium salts (Lithobid, Eskalith, Eskalith ER).

3. A method according to claim 1 wherein depressive disorders are treated with medications that reduce neurotransmission by blocking monoamine and other receptors that are involved in the transmission process. Such medications include, but are not limited to risperidone (Risperdal), olanzapine, (Zyprexa), quetiapine fumarate (Seroquel), ziprasidone (Geodon), aripiprazole (Abilify), clozapine (Clozaril), pimozide (Orap), fluphenazine (Prolixin), halopiradol (Haldol), thiothixine (Navane), trifluoperazine (Stellazine), mesoridazine (Serentil), prochlorperazine (Compazine), molindone (Moban), thioridizine (Mellaril), and chlorpromazine (Thorazine).

4. A method according to claim 1 wherein depressive disorders are treated with medications that reduce monoamine neurotransmission by directly or indirectly affecting voltage-gated sodium and/or calcium channels.

5. A method according to claim 1 wherein depressive disorders are treated with medications that reduce neurotransmission by inhibiting the synthesis and/or release of monoamines by the neuron.

6. A method according to claim 1 wherein depressive disorders are treated with medications that reduce neurotransmission by facilitating monoamine reuptake.

7. A method according to claim 1 wherein depressive disorders are treated with medications that reduce neurotransmission by increasing the breakdown of monoamines inside or outside of the neuron.

8. A method according to claim 1 wherein depressive disorders are treated with medications that reduce neurotransmission by increasing inhibitory input to neurons in dysphoria-producing pathways.

9. A method according to claim 1 wherein depressive disorders are treated with medications that directly or indirectly increase the activity of gamma-aminobutyric acid (GABA) in the brain, i.e., by increasing GABA synthesis and/or release, or by inhibiting GABA reuptake.

10. Any method by which depressive disorders are treated by reducing neurontransmission in the brain.

11. A method according to claim 1 wherein depressive disorders are treated by combining, in a single formulation (tablet, capsule, or liquid), an antidepressant such as citalopram (Celexa), escitalopram oxalate (Lexapro), fluoxetene (Prozac), sertraline (Zoloft), paroxetine (Paxil, Paxil CR), fluvoxamine (Luvox), nefazodone (Serzone), bupropion (Wellbutrin, Wellbutrin XL), mirtazepine (Remeron), venlafaxine (Effexor, Effexor XR), or other antidepressant, with one or a combination of the medications listed in claim 2.

12. A method according to claim 1 wherein depressive disorders are treated by combining, in a single formulation (tablet, capsule, or liquid), an antidepressant such as citalopram (Celexa), escitalopram oxalate (Lexapro), fluoxetene (Prozac), sertraline (Zoloft), paroxetine (Paxil, Paxil CR), fluvoxamine (Luvox), nefazodone (Serzone), bupropion (Wellbutrin, Wellbutrin XL), mirtazepine (Remeron), venlafaxine (Effexor, Effexor XR), or other antidepressant, with one or a combination of the medications listed in claim 3.

13. A method according to claim 1 wherein depressive disorders are treated by combining, in a single formulation (tablet, capsule, or liquid), an antidepressant such as citalopram (Celexa), escitalopram oxalate (Lexapro), fluoxetene (Prozac), sertraline (Zoloft), paroxetine (Paxil, Paxil CR), fluvoxamine (Luvox), nefazodone (Serzone), bupropion (Wellbutrin, Wellbutrin XL), mirtazepine (Remeron), venlafaxine (Effexor, Effexor XR), or other antidepressant, with one or more of the compounds from each of the two groups listed in claims 2 and 3, respectively.

14. A method according to claim 1 wherein depressive disorders are treated by combining, in a single formulation (tablet, capsule, or liquid), two or more of the compounds listed in claim 2.

15. A method according to claim 1 wherein depressive disorders are treated by combining, in a single formulation (tablet, capsule, or liquid), two or more of the compounds listed in claim 3.

16. A method according to claim 1 wherein depressive disorders are treated by combining, in a single formulation (tablet, capsule, or liquid), one or more of the compounds listed in claim 2, with one or more of the compounds listed in claim 3.
